(19) United States
(12) Patent Application Publication
(10) Pub. No.: US 2010/0174101 A1
(43) Pub. Date: Jul. 8, 2010

(54) Process for the manufacture of 7-alpha-[9-(4,4,5,5,5-penta fluoropentylsulphinyl) nonyl]estra-1,3,5-(10)- triene-3,17-beta-diol

(75) Inventors: Suoding Cao, Shaanxi (CN); Yuanlin Zhou, Shaanxi (CN); Yaping Shen, Shaanxi (CN); Tao Chen, Shaanxi (CN)

Correspondence Address: HARNESS, DICKEY & PIERCE, P.L.C., P.O. BOX 828, BLOOMFIELD HILLS, MI, 48303, US

(73) Assignee: XI'AN LIBAND PHARMACEUTICAL CO., LTD., Shaanxi (CN)

(21) Appl. No.: 12/532,444

(22) PCT Filed: Sep. 24, 2007
(86) PCT No.: PCT/CN2007/070760

§ 371 Date: Sep. 22, 2009

(51) Int. Cl. C07J 31/00

(52) U.S. Cl. 552/626

(57) ABSTRACT

The present invention provides a novel multi-step process for the manufacturing Fulvestrant, which is economical and convenient to operate at commercial scale, and requires only simple chromatographic separations after the coupling step of adding the side chain to the 7 position of the steroid.

### TECHNICAL FIELD

The present invention relates to a process for the preparation of 7-alpha-[9-(4,4,5,5,5-penta fluoropentylsulphinyl)nonyl]estra-1,3,5-(10)-triene-3,17-beta-diol (Fulvestrant) and the novel intermediates for use in the process.

### BACKGROUND OF THE INVENTION

7-alpha49-(4,4,5,5,5-penta fluoropentylsulphinyl)nonyl]estra-1,3,5-(10)-triene-3,17-beta-diol (Fulvestrant) belongs to a class of antioestrogens. It blocks the effect of estrogen in the body by binding to and decreasing estrogen receptors in the cells, Fulvestrant is used to treat some types of metastatic breast cancer that require estrogen to grow, in postmenopausal women whose cancer has progressed following treatment with other antiestrogen medication.

U.S. Pat. No. 4,659,516 describes a process for the preparation of Fulvestrant. In this process, the expensive dienone intermediate has been prepared in seven steps involving tedious and complex chemistry, result in low yields, and require time consuming cumbersome chromatographic separations. The step of adding the side chain to the 7 position of the dienone steroid is favorable to the preferred a position, however the selectivity is poor, the α/β ratio is 1.9:1. This process is also published in the literature (Bowlers J. Steroids, (1989) 71-79).

WO 02/32922 A1 describes an improved process for the preparation of Fulvestrant. In this process, there are only four steps from the dienone intermediate, however the selectivity in the coupling step is still poor, the α/β ratio is only improved to about 2.5:1, the unwanted β form isomer need to be removed in the final step by special purification procedure.

### SUMMARY OF THE INVENTION

The present invention provides a novel multi-step process for the manufacturing Fulvestrant which is economical, convenient to operate at commercial scale, and requires only simple chromatographic separations after the coupling step of adding the side chain to the 7 position of the steroid.

According to the present invention, Fulvestrant 8 is manufactured by the reaction sequence shown in the following schemes (scheme 1 and scheme 2).

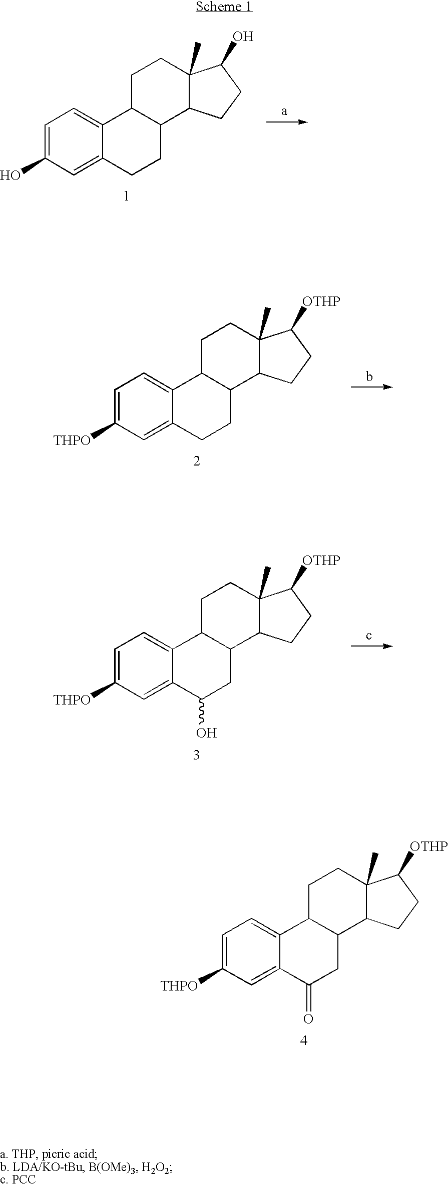

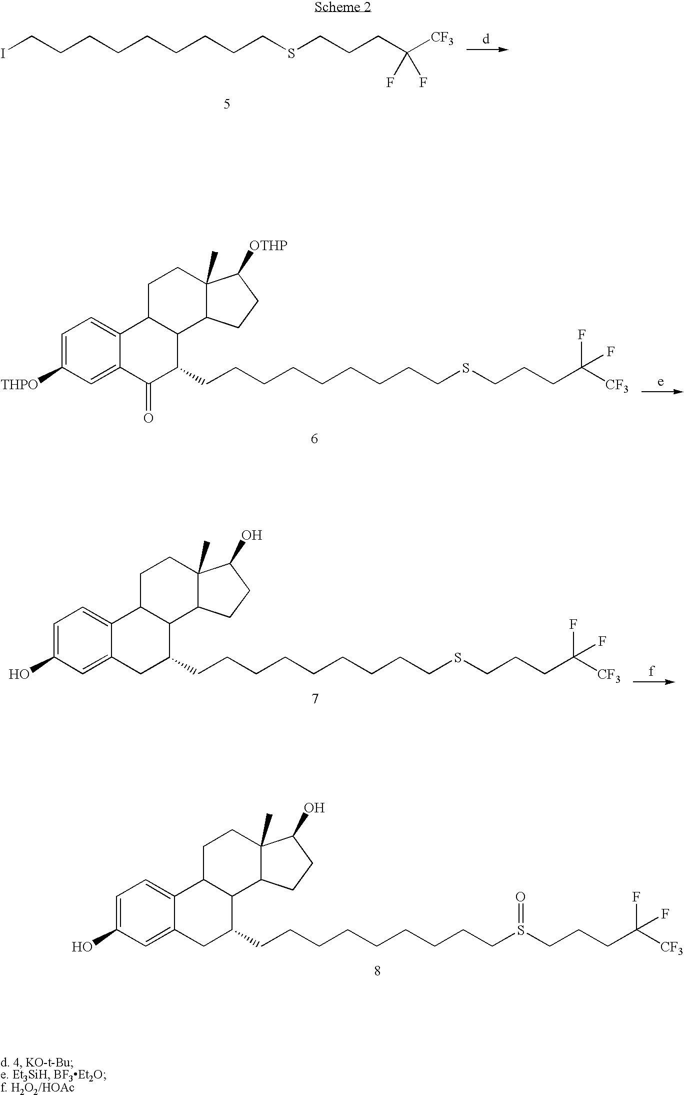

### DETAILED DESCRIPTION OF THE INVENTION

In accordance with the present invention, Fulvestran 8 is manufactured starting from commercially available β-Estradiol 1. The hydroxyl groups of β-Estradiol are first protected as ethers, in this specific case, the hydroxyl groups are protected as tetrahydropyranyl ether.

The compound 2 is then deprotonated under superbase condition using potassium ter-butoxide (KO-tBu) and lithium diisopropylamide (LDA) as reagents and the resulting anion is treated with trimethyl borate, followed by hydrogen peroxide to afford the alcohol 3. This alcohol is further oxidized using an oxidation agent such as pyridinium chlorochromate (PCC) or sodium hypochlorite to give ketone 4.

Introduction of a 7α side-chain is accomplished in good yield by deprotonated of ketone 4 with either potassium t-amylate or KO-tBu in dry tetrahydrofuran, followed by quenching the resulting enolate with compound 5. Compound 6 is obtained as a single epimer, and the product can be readily separated by a short column chromatography from the unwanted O-alkylation product.

Compound 6 is thereafter deoxygenated by treatment of Compound 6 with BF₃.Et₂O/Et₃SiH in dichloromethane, under this condition, the acid sensitive protecting groups are also removed to provide desired compound 7.

The compound 7 is then oxidized using hydrogen peroxide in tetrahydrofuran to give Fulvestrant 8 in good yield.

The following examples illustrate the present invention and as such are not to be considered as limiting the invention set forth in the claims appended hereto.

Synthesis of Compound 2

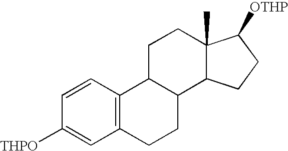

To a mixture of estradiol (1) (24.5 g, 89.9 mmol) and picric acid (50 mg) in toluene (300 mL) was added 3,4-dihydro-2H-pyran (70 mL). The reaction mixture was heated to reflux until TLC indicated the completion of the reaction. The solution was cooled to room temperature, washed with saturated NaHCO₃(100 mL) and saturated NaCl (100 mL), and dried over MgSO₄. Solvent was removed under reduced pressure. Tetrahydrofuran (30 mL) was added and solvent was removed under reduced pressure. The obtained crude product of compound 2 was used for next reaction without further purification.

Synthesis of Compound 3

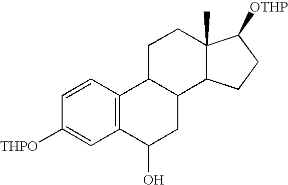

To a solution of diisopropylamine (54 mL, 380 mmol) in dry tetrahydrofuran (100 mL) at −78° C. was added n-BuLi (144 mL, 2.5 M in hexane, 360 mmol), followed by KOt-Bu in tetrahydrofuran made from dissolving solid KOt-Bu (40.4 g, 360 mmol) in 400 mL of dry tetrahydrofuran, a solution of compound 2 (crude product, 89.9 mmol) in tetrahydrofuran (80 mL) was added slowly. The resulting dark red solution was stirred at −78° C. for 3.5 h. Trimethylborate (80 mL) was slowly added. The reaction was then slowly warmed to 0° C. and was stirred at 0° C. for 2 h. To the solution, 30% H₂O₂ (200 mL) was added slowly and it was stirred at room temperature for 2 h. The reaction was then re-cooled to 0° C. and 25% Na₂S₂O₃ (1400 mL) was slowly added. The solution was stirred at 0° C. for 1 h, and extracted with ethyl acetate (2×300 mL). The combine extract was dried over MgSO₄. Evaporation of solvent afforded crude compound 3 as a pale yellow syrup.

Synthesis of Compound 4

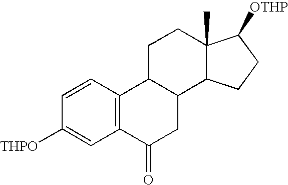

To a suspension of compound 3 (21 g, 46 mmol) and K₂CO₃ (3 g) in CH₂Cl₂ (200 mL) was added a mixture of pyridinium chlorochromate (25 g, 115 mmol) and Celite (30 g) over a period of 30 min. After reaction was complete, the reaction mixture was immediately loaded on the column (100 g of silica gel). Column was eluted with hexane (150 mL), then with 10% ethyl acetate in hexane (90% hexane, 10% ethyl acetate). Removal of solvent under reduced pressure gave compound 4.

Synthesis of Compound 6

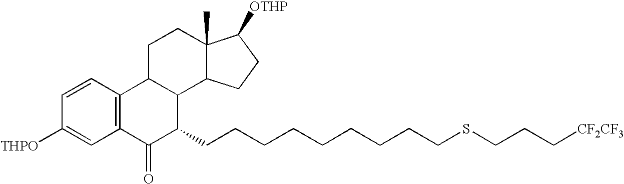

To a solution of compound 4 (12 g, 26.4 mmol) in tetrahydrofuran (150 mL) was added KO-tBu (35 mL, 1 M solution in tetrahydrofuran, 10 mmol) at 0° C. and the solution was stirred at 0° C. for 75 min. The reaction mixture was then cooled to −70° C. Compound 5 (20 g, ˜88%, 39.4 mmol) in tetrahydrofuran (50 mL) was added slowly. The reaction mixture was stirred at −70° C. to 0° C. about 15 h (overnight). The reaction mixture was participated with 300 ml of ethyl acetate and 300 ml of water. The aqueous layer was extracted with ethyl acetate (200 mL and 150 ml). The combined organic layers were dried over Na₂SO₄ and concentrated to give crude product (˜20 g) as brownish oil. The crude product was subjected to a silica gel chromatography by using 5%-15% ethyl acetate in hexane as eluents to give compound 6 (8.82 g, 43.1%).

Synthesis of Compound 7

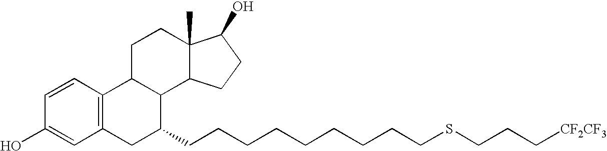

To a solution of compound 6 (8.8 g, 11.38 mmol) in dichloromethane (440 mL) was added Et₃SiH (88 mL). The reaction was cooled to 0° C. BF₃.Et₂O (176 mL) was added dropwise. After the addition was complete (˜14 h). The mixture was extracted with ethyl acetate (250 ml, 200 ml and 150 ml). The combined extract was washed with saturated brine (200 ml×2) and dried over Na₂SO₄. Solvent was evaporated to give a crude product. The crude product was purified by a short silica gel chromatography using ethyl acetate/Hexane 1:1 as eluents to give pure compound 7 (5.45 g, 81%).

Synthesis of Compound 8

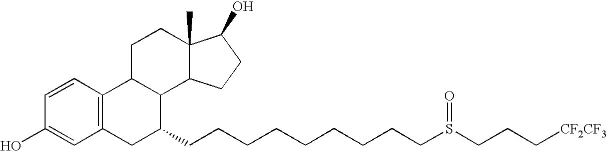

To a solution of compound 7 (3.2 g, 5.42 mmol) in ethyl acetate (7.5ml) and acetic acid (1.96 ml) was slowly added H₂O₂ (17%, which was made from 35% commercial H₂O₂ dilute half with water, 3.47 ml) over 25 min. After the reaction was complete (˜6-8 h), the reaction was quenched with 25% Na₂S₂O₃ slowly. Ethyl acetate (100 ml) was added to dilute the mixture. Organic layer was separated and the aqueous layer was extracted with ethyl acetate (100 ml×2).

The combined organic layers were dried over Na₂SO₄ and concentrated to give formed crude product. The product was purified by silica gel chromatography using 2:1 ethyl acetate/hexane as eluents to provide compound 8 (2.70 g, 85%).

1. A process for preparing compound 8 or a pharmaceutically acceptable salt thereof, 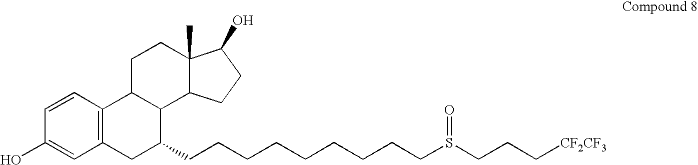
Wherein
the process comprising:
(a) protecting hydroxyl groups at compound 1 as tetrahydropyranyl ether to give compound 2; 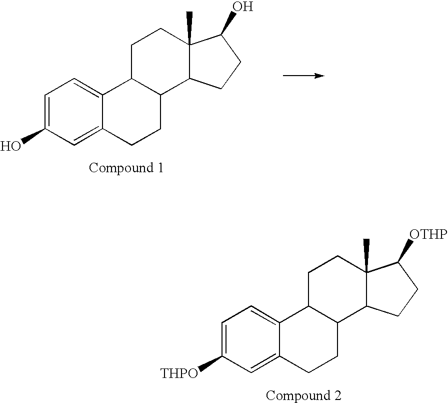
(b) reacting compound 2 with bases, followed by treating with trimethyl borate and hydrogen peroxide to give compound 3

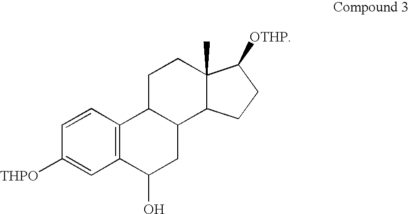

2. The process of claim 1, wherein the bases are potassium ter-butoxide and lithium diisopropylamide.

3. The process of claim 1, which is conducted as a continuous process.

4. The process of claim 1, further including oxidizing compound 3 with an oxidation agent to give compound 4

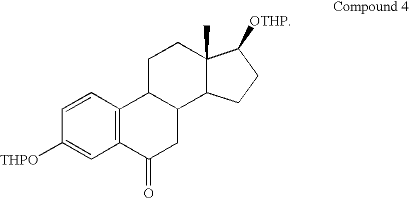

5. The process of claim 4, wherein the oxidation agent is pyridinium chlorochromate or pyridinium dichromate or sodium hypochlorite.

6. The process of claim 1, further including reacting compound 4 with compound 5 to form compound 6 as single epimer,

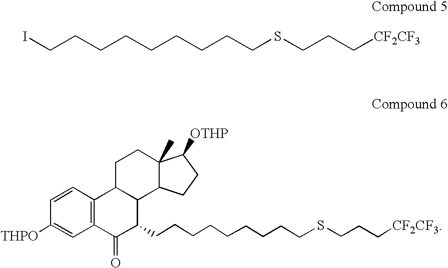

7. The process of claim 6, wherein the reaction is performed at 0° C. to −78° C.

8. The process of claim 6, wherein compound 6 is further purified by column chromatography.

9. The process of claim 1, wherein compound 6 is deoxygenated by treatment of compound 6 with BF₃.Et₂O/Et₃SiH in a solvent.

10. The process of claim 9, wherein the solvent is a mixture of acetic acid and ethyl acetate.
